(19) United States
(12) Patent Application Publication
(10) Pub. No.: US 2025/0331725 A1
(43) Pub. Date: Oct. 30, 2025

(54) MULTIPLE SENSOR INTRACARDIC DEVICES FOR CROSS VALVE MEASUREMENTS AND/OR POSITION TRACKING AND ASSOCIATED SYSTEMS AND METHODS

(71) Applicant: KONINKLIJKE PHILIPS N.V., EINDHOVEN (NL)

(72) Inventors: Thomas John McPEAK, CALEDONIA, MN (US); James JENSEN, MAPLE GROVE, MN (US); Nicholas Andrew STROM, MAPLE GROVE, MN (US); Alyssa TORJESEN, EXETER, NH (US)

(21) Appl. No.: 19/194,498

(22) Filed: Apr. 30, 2025

Related U.S. Application Data
(60) Provisional application No. 63/640,233, filed on Apr. 30, 2024.

(51) Int. Cl. A61B 5/021; A61B 5/00; A61B 5/06

(52) U.S. Cl. CPC A61B 5/02141; A61B 5/066; A61B 5/6847; A61B 5/6869; A61B 5/743; A61B 2560/0468; A61B 2562/0247

(57) ABSTRACT

Intracardiac devices (e.g., guidewires, catheters, sheaths) with multiple sensors for use in cardiac valve replacement, structural heart procedures, and other medical procedures are provided. The multiple sensors allow for measurements to be made at multiple locations (e.g., on both sides of a heart valve) without having to move the intracardiac device as well as facilitate tracking of the intracardiac device within the anatomy of the patient. In some aspects, the intracardiac devices include one or more electrodes for pacing a heartbeat of the patient, including while obtaining measurements (e.g., pressure measurements) with the multiple sensors. Associated systems and methods are also provided.

### TECHNICAL FIELD

The subject matter described herein relates to intracardiac devices (e.g., guidewires, catheters, sheaths) with multiple sensors for use in cardiac valve replacement, structural heart procedures, and other medical procedures. The multiple sensors allow for measurements to be made at multiple locations (e.g., on both sides of a heart valve) without having to move the intracardiac device as well as allowing for the tracking of the intracardiac device within the anatomy of the patient.

### INTRODUCTION

One common type of valve disease is aortic valve stenosis. With the advent of minimally invasive procedures, valve replacement is becoming a more common therapy. For example, transvenous/transcatheter aortic valve replacement (TAVR) is becoming more frequently used as technology and doctor skillsets improve. This minimally invasive approach to valve replacement is an alternative to open heart surgical aortic valve replacement (SAVR). One difference between a TAVR procedure and a SAVR procedure is, in the SAVR procedure, the natural aortic valve is removed during an open-heart procedure that is performed once, i.e. when the natural aortic valve is replaced. In a TAVR procedure, the damaged valve, whether the valve is a natural aortic valve or a previous SAVR or TAVR valve, is left in place. These valves may have anatomical abnormalities, calcification, or infection. Inserting a new valve may cause complications in the TAVR procedure, including valve migration, valve embolization, paravalvular leakage, patient-prosthesis mismatch, and blockage of the coronary arteries restricting blood flow to the heart.

TAVR has been approved for low-risk patients, which in general are of younger age and live longer. Recent studies have shown that the life of a TAVR valve will be on average 8 years, so there will be an increase in replacement TAVR procedures. Old TAVR leaflets may be fibrosed, calcified, and/or thickened over time creating a barrier to the replacement valve. The old leaflets also pose a more serious risk of coronary obstruction than native valves. During a heart valve replacement or other intracardiac procedure, gripping a heart valve leaflet from the center of the valve annulus towards the root of the leaflet (or vice versa) can be advantageous for several reasons, including resection of the leaflet. A transcatheter edge to edge repair (TEER) procedure, used to treat mitral valve regurgitation, is a procedure in which the leaflets of the mitral valve are “grabbed” by a clip like device, and then drawn together as the device is deployed.

In some instances, removing old valve leaflets helps prepare the implant site for a cleaner valve deployment and operation. Removal of the leaflets may introduce issues with aortic regurgitation or aortic insufficiency in the patient. This is especially important in valve-in-valve TAVR procedure as the risk for coronary obstruction is higher. During the implantation of a replacement valve during a TAVR procedure, the action of expanding the replacement valve may push the current leaflets to the sides of the aorta and cover the openings to the coronary arteries, reducing oxygenated blood to the heart. In some instances, a laser sheath and balloon catheter assembly device may be used to remove parts of the aortic valve leaflets prior to deploying a replacement valve.

There are a variety of procedures during which an interventionalist would like to know the pressures at various points within the heart chamber(s), for example, during a percutaneous valvular implant, where the ability to pace the heart may also be advantageous. Currently, multiple devices-beyond any treatment devices-are necessary to even attempt to obtain such information. For example, an invasive blood pressure transducer system and/or a pressure-sensing guidewire or catheter with an integral pressure transducer may be utilized to obtain pressure measurements and, even then, may only provide pressure measurements at one or two specific locations. Further, a separate pacing lead and/or wire may be required for pacing.

The information included in this Introduction section of the specification, including any references cited herein and any description or discussion thereof, is included for context and/or technical reference purposes only and is not to be regarded as subject matter by which the scope of the disclosure is to be bound or otherwise limited in any manner.

### SUMMARY

Disclosed are multiple sensor devices (e.g., guidewires, catheters, and/or sheaths) and associated systems and methods for use in cardiac valve replacement, structural heart procedures, and other medical procedures. The multiple sensor devices and associated systems and methods of the present disclosure provide a plurality of pressure sensors and at least one electrode. The plurality of pressure sensors may provide static pressure measurements at a plurality of locations along the length of the multiple sensor device. In addition, the plurality of pressure sensors may also be utilized for position and/or orientation tracking of the multiple sensor device within the anatomy of the patient. Further, the electrode(s) of the multiple sensor device may be utilized to pace the heartbeat of the patient. In some instances, the electrode(s) may pace the heartbeat of the patient while the static pressure measurements are obtained by the plurality of pressure transducers. The multiple sensor devices of the present disclosure provide numerous advantages over existing approaches, including eliminating the need for multiple different catheters and/or guidewires to obtain a plurality of pressure measurements at different locations within the heart and/or associated vasculature, providing pacing, providing position tracking functionality, streamlining procedural workflows, etc.

In some aspects, a system is provided. The system may include a multiple sensor device and a processing system in communication with the multiple sensor device. The multiple sensor device may include a flexible elongate member with a distal portion sized and shaped for advancement through a vessel of a patient and at least partially through a valve of the patient. The flexible elongate member may have an outer diameter between 0.014″ and 0.092″ in some instances. The distal portion may include a plurality of pressure sensors and at least one electrode. The plurality of pressure sensors may be spaced apart along a length of the distal portion. The plurality of pressure sensors may comprise optical pressure sensors, electrical pressure sensors, and/or piezoelectric pressure sensors. The electrode(s) may be configured to pace a heartbeat of the patient. The processing system may be configured to receive, from the plurality of pressure sensors, signals indicative of static pressure measurements obtained by the plurality of pressure sensors. The static pressure measurements may include a first static pressure measurement obtained by a first pressure sensor positioned on a first side of the valve and a second static pressure measurement obtained by a second pressure sensor positioned on a second side of the valve opposite the first side of the valve. The processing system may be further configured to output, to a display in communication with the processing system, an indication of a pressure differential between the first static pressure measurement and the second static pressure measurement. The pressure differential may include a numerical value representative of a difference between the first static pressure measurement and the second static pressure measurement, the first static pressure measurement and the second static pressure measurement, and/or be color coded.

In some aspects, the processing system is further configured to receive positioning signals from the plurality of pressure sensors, wherein the positioning signals are based on ultrasound positioning signals transmitted by an ultrasound imaging device. The ultrasound imaging device may include a transesophageal echocardiography (TEE) device, a transthoracic echocardiography (TTE) device, or a intracardiac echocardiography (ICE) device. Based on the received positioning signals, the processing system determines locations of the plurality of pressure sensors within the patient and outputs, to an imaging display in communication with the processing system, an indication of a position of the distal portion of the multiple sensor device within the patient based on the determined locations of the plurality of pressure sensors. In some aspects, the indication of the position of the distal portion of the multiple sensor device within the patient includes an overlay on an ultrasound image of anatomy of the patient based on ultrasound data obtained from the ultrasound imaging device. The processing system may be further configured to output, to the imaging display, an indication of an orientation of the distal portion of the multiple sensor device within the patient based on the determined locations of the plurality of pressure sensors. In some aspects, the processing system may be further configured to filter the signals received from the plurality of pressure sensors indicative of the static pressure measurements obtained by the plurality of pressure sensors from the positioning signals received from the plurality of pressure sensors.

In some aspects, a method is provided. The method may include receiving, by a processing system from a multiple sensor device positioned within a patient, positioning signals from a plurality of pressure sensors spaced apart along a length of a distal portion of the multiple sensor device. The positioning signals may be based on ultrasound positioning signals transmitted by an ultrasound imaging device. The method may also include outputting, to an imaging display in communication with the processing system, an indication of a position of the distal portion of the multiple sensor device within the patient based on locations of the plurality of pressure sensors determined based on the received positioning signals.

The method may also include receiving, by the processing system from the plurality of pressure sensors, signals indicative of static pressure measurements obtained by the plurality of pressure sensors. The static pressure measurements may include a first static pressure measurement obtained by a first pressure sensor positioned on a first side of a valve and a second static pressure measurement obtained by a second pressure sensor positioned on a second side of the valve opposite the first side of the valve. The method may also include outputting, to a display in communication with the processing system, an indication of a pressure differential between the first static pressure measurement and the second static pressure measurement.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of aspects of the present disclosure, e.g., as defined in the claims, is provided in the following written description of various examples and/or aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1A illustrates a view of a human heart according to aspects of the present disclosure.

FIG. 1B is a cross-sectional view of a human heart according to aspects of the present disclosure.

FIG. 2 is a side view of a multiple sensor device positioned within patient anatomy according to aspects of the present disclosure.

FIG. 3 is a cross-sectional view of an aortic valve replacement in a human heart according to aspects of the present disclosure.

FIG. 4A is a cross-sectional view of a human heart undergoing a mitral valve transcatheter edge-to-edge repair (TEER) procedure, according to aspects of the present disclosure.

FIG. 4B is a close-up view of the TEER procedure of FIG. 4A, according to aspects of the present disclosure.

FIG. 5 is a schematic diagram of a system according to aspects of the present disclosure.

FIG. 6 is a schematic diagram of a processing system according to aspects of the present disclosure.

FIG. 7A is a diagrammatic cross-sectional view of a distal portion of a multiple sensor device positioned across an aortic valve in an open state according to aspects of the present disclosure.

FIG. 7B is a diagrammatic cross-sectional view of a distal portion of a multiple sensor device positioned across an aortic valve in a closed state according to aspects of the present disclosure.

FIG. 8A is a diagrammatic cross-sectional view of a distal portion of a multiple sensor device positioned across an aortic valve in an open state according to aspects of the present disclosure.

FIG. 8B is a diagrammatic cross-sectional view of a distal portion of a multiple sensor device positioned across an aortic valve in a closed state according to aspects of the present disclosure.

FIG. 9A is a cross-sectional view of a human heart being imaged by an intracardiac echography (ICE) catheter for tracking positioning of a distal portion of a multiple sensor device, according to aspects of the present disclosure.

FIG. 9B is a cross-sectional view of a human heart being imaged by an intracardiac echography (ICE) catheter for tracking positioning of a distal portion of a multiple sensor device similar to that of FIG. 9A but showing the distal portion of the multiple sensor device in a different location, according to aspects of the present disclosure.

FIG. 10A is a cross-sectional view of a human heart being imaged by an ultrasound device for tracking positioning of a distal portion of a multiple sensor device, according to aspects of the present disclosure.

FIG. 10B is a cross-sectional view of a human heart being imaged by an ultrasound device for tracking positioning of a distal portion of a multiple sensor device similar to that of FIG. 10A but showing the distal portion of the multiple sensor device in a different location, according to aspects of the present disclosure.

FIG. 11 is a flow diagram of a method of utilizing a multiple sensor device according to aspects of the present disclosure.

### DETAILED DESCRIPTION

For the purpose of promoting an understanding of the principles of the present disclosure, reference will now be made to the examples illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one example and/or aspect may be combined with the features, components, and/or steps described with respect to other examples and/or aspects of the present disclosure. Additionally, while the description below may refer to blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

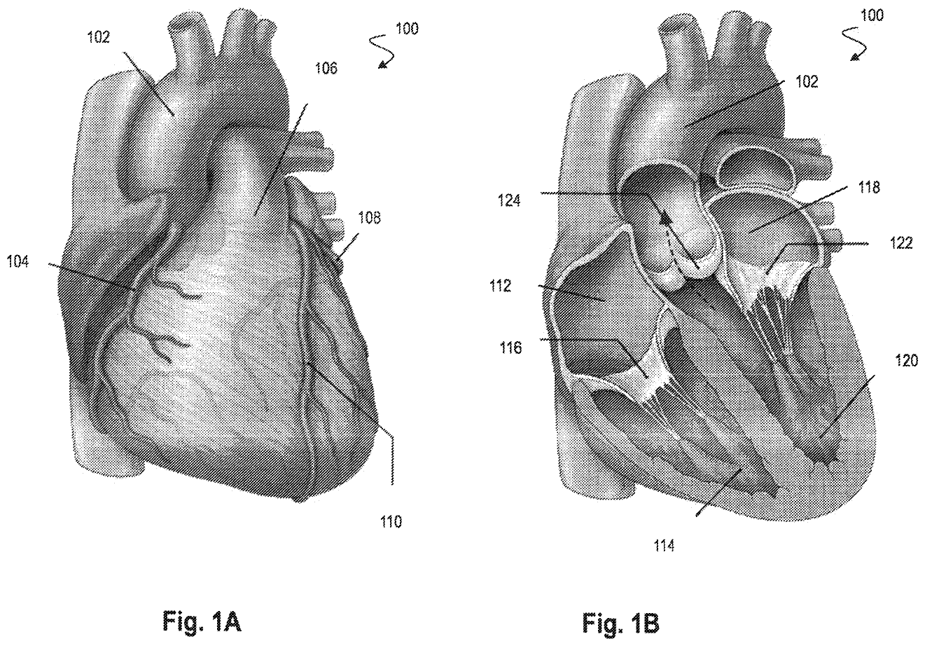

Referring to FIG. 1A, shown is a view of a human heart 100 according to aspects of the present disclosure. Visible are an aorta 102 from which stems a right coronary artery 104 and a left main coronary artery 106. The left main coronary artery 106 branches into a left circumflex coronary artery 108 and a left anterior descending coronary artery 110. The right coronary artery 104, the left main coronary artery 106, the left circumflex coronary artery 108, and a left anterior descending coronary artery 110 are the arteries that provide oxygen-rich blood to muscles of the human heart 100.

FIG. 1B is a cross-sectional view of the human heart 100 according to aspects of the present disclosure. Visible are a right atrium 112 and a right ventricle 114. In that regard, oxygen-poor blood enters the human heart 100 in the right atrium 112 and travels to the right ventricle 114 through the tricuspid valve 116. The oxygen-poor blood leaves the right ventricle 114 and travels to the lungs. Also visible are a left atrium 118 and a left ventricle 120. In that regard, oxygen-rich blood is received from the lungs in the left atrium 118 and travels to the left ventricle 120 through the mitral valve 122. The oxygen-rich blood leaves the left ventricle 120 and goes out to the body through the aorta 102 via an aortic valve 124.

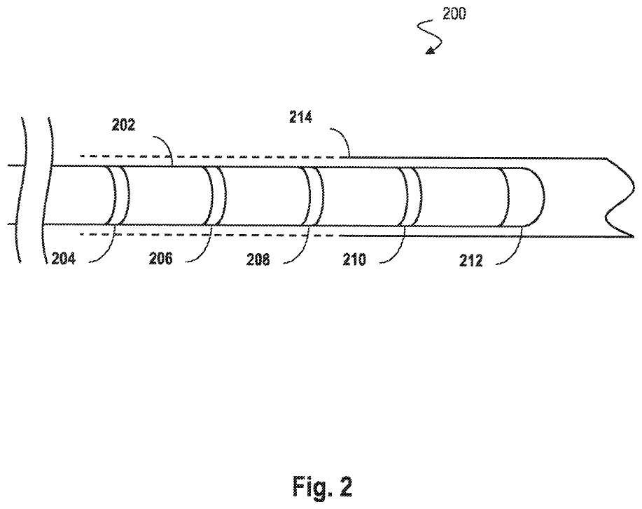

FIG. 2 is a side view of a multiple sensor device 200 positioned within patient anatomy according to aspects of the present disclosure. In particular, a distal portion of the multiple sensor device 200 is shown positioned within a vessel 214 (e.g., artery, vein, organ, tubular structure, cavity, etc.) of a patient. The multiple sensor device 200 includes an elongate member 202. As used herein, “elongate member” or “flexible elongate member” may include at least any long, flexible structure that can be inserted into the vessel 214 of a patient. While the illustrated embodiments of the “elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, catheters, guidewires, and/or sheaths. In this regard, catheters may or may not include a lumen extending along its length for receiving and/or guiding other instruments. If the catheter includes a lumen, the lumen may be centered or offset with respect to the cross-sectional profile of the device.

The elongate member 202 includes a plurality of sensors (e.g., sensors 204, 206, 208, and 210) disposed along the length of the elongate member 202. The elongate member 202 may include any suitable number of sensors, including without limitation 2, 3, 4, 5, 6, 7, 8, 9, 10, or otherwise. In various aspects, the sensors 204, 206, 208, and 210 may include a common type of sensor (e.g., all the same sensor and/or same type of sensor). For example, in some aspects each of the sensors 204, 206, 208, and 210 is a pressure sensor. The pressure sensor may be any type suitable for use within the elongate member 202, including without limitation an electrical pressure sensor (e.g., piezoelectric, MEMS, Wheatstone bridge, etc.), an optical pressure sensor, or otherwise. In other aspects, the sensors 204, 206, 208, and 210 may include multiple types of sensors (e.g., two or more different sensors and/or type(s) of sensors). In some aspects, the sensors 204, 206, 208, and 210 may include one or more sensors, including transducers, corresponding to sensing modalities such as pressure, flow, IVUS, OCT, other suitable modalities, and/or combinations thereof.

In some embodiments, the elongate member 202 may include one or more electrodes (e.g., electrode 212) disposed at and/or near a distal end of the elongate member. The electrode(s) may take any suitable form, including without limitation a coil, a ring, a plate, etc. The elongate member 202 may include any suitable number of electrodes, including without limitation 1, 2, 3, 4, 5, 6, 7, 8, 9, 10, or otherwise. In some aspects, the electrode(s) are configured to pace a heartbeat of the patient. In this regard, the electrode(s) may be placed in contact with and/or close proximity to tissue of the heart of the patient and electrical current selectively passed through the electrode(s) to the tissue to pace the heartbeat of the patient in a desired manner.

The sensors and the electrodes and associated communication lines (e.g., electrical and/or optical) may be sized and shaped to allow for the diameter of the elongate member 202 to take the form of a guidewire, catheter, or sheath. For example, an outer diameter of the elongate member 202, such as a guidewire, catheter, or sheath, containing the sensors and the electrodes as described herein may be between about 0.014″ (0.3556 mm) and about 36.0 F (0.47″, 11.938 mm), with some particular embodiments having outer diameters of approximately 0.014″ (0.3556 mm), approximately 0.018″ (0.4572 mm),.035″ (0.889 mm), 3.0 F (.039″, 1.00 mm), 3.2 F (0.042″, 1.07 mm), 3.5 F (.046″, 1.17 mm), 7.0 F (0.092″, 2.333 mm), or otherwise. As such, the elongate members 202 of the present application may be suitable for use in a wide variety of lumens within a human patient besides those that are part or immediately surround the heart, including veins and arteries of the extremities, renal arteries, blood vessels in and around the brain, and other lumens.

As shown in FIG. 2, a distal portion of the elongate member 202 including the sensors and the electrode may be advanced through a vessel 214 of a patient. The vessel 214 represents fluid filled or surrounded structures, both natural and man-made, within a living body and can include for example, but without limitation, structures such as: organs including the liver, heart, kidneys, gall bladder, pancreas, lungs; ducts; intestines; nervous system structures including the brain, dural sac, spinal cord and peripheral nerves; the urinary tract; as well as valves within the blood or other systems of the body. In addition to natural structures, elongate member 202 may be used to examine man-made structures such as, but without limitation, heart valves, stents, shunts, filters, and/or other devices positioned within the body, for example, other guidewires, catheters, delivery sheath, treatment devices, and/or deployment devices.

When the sensors and/or electrodes are in use, a communication channel, a power channel, and/or an activation channel may couple the sensors and/or electrodes to a processing system. The communication channel, the power channel, and/or the activation channel may include an electrical conductor, a plurality of electrical conductors (e.g., a cable or bundle), an optical fiber, a plurality of optical fibers, and/or a wireless transceiver, and/or combinations thereof. The communication channel, the power channel, and/or the activation channel may extend along the length of the elongate member 202 from a distal portion (e.g., where the sensors and/or electrodes are positioned) to a proximal portion (e.g., where a handle and/or connector may be positioned). The communication channel, the power channel, and/or the activation channel may communicate signals and/or data to and/or from the sensors and/or the electrodes.

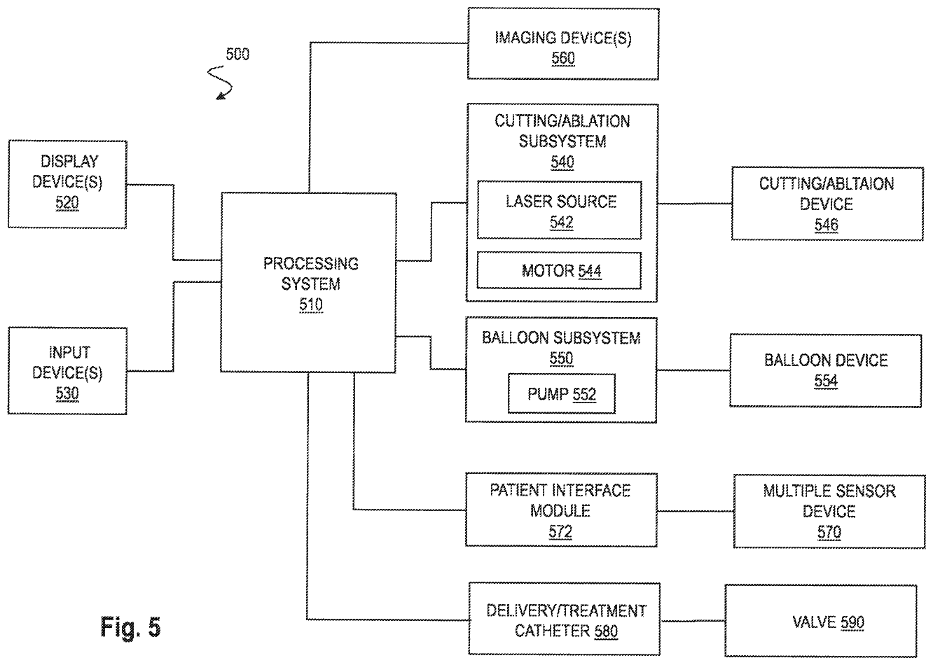

In some aspects, a patient interface monitor (PIM) (e.g., PIM 572 of FIG. 5) may be coupled to a proximal portion of the elongate member 202. The PIM may be operable to receive medical sensing data collected using the sensors and/or the electrodes. The PIM may be operable to pre-process and/or process the received data and may transmit the received and/or pre-processed/processed data to a processing system. For example, in some embodiments the PIM performs preliminary processing of the sensing data prior to transmitting the pre-processed data to the processing system. In this regard, the PIM may perform amplification, filtering, time-stamping, identification, and/or aggregating of the data. The PIM may also transfer data such as commands and/or activation signals from the processing system to the sensors and/or the electrode(s) of the elongate member 202. In some instances, these commands may include commands to enable and disable one or more of the sensors, to configure modes of operation for one or more of the sensors, enable and disable one or more of the electrodes, to configure modes of operation for one or more of the electrodes, and/or combinations thereof. In some embodiments, the PIM may also supply power to drive the operation of the sensors and/or electrodes.

The PIM (e.g., PIM 572 of FIG. 5) may be communicatively coupled to a processing system (e.g., processing system 510 of FIG. 5). The processing system may control operation and/or data acquisition of the sensors and/or electrodes of the elongate member 202, perform data processing, perform data interpretation, provide a user interface(s) and controls, and/or provide associated displays. For example, the processing system may receive data from the sensors and/or electrode(s) of the elongate member 202 (directly or via the PIM), process the data to render it suitable for display, and present the processed data at a user display (e.g., display device(s) 520 of FIG. 5).

In some aspects, the processing system may receive signals indicative of static pressure measurements obtained by a plurality of pressure sensors of the elongate member 202. Static pressure measurements may be viewed in contrast to stagnation pressure measurements. In this regard, stagnation pressure measurements may include and/or be affected by blood flow. For example, invasive blood pressure transducer systems that rely on fluid column pressure measurements and are often used in structural heart procedures provide the stagnation that can include additional pressure or less pressure as a result of the momentum of flowing blood. In contrast, static pressure measurements may be obtained by the pressure sensors of the elongate member 202 in a direction substantially perpendicular to the longitudinal axis of the elongate member and, therefore, substantially perpendicular to the direction of blood flow through the vessel in which the elongate member is positioned.

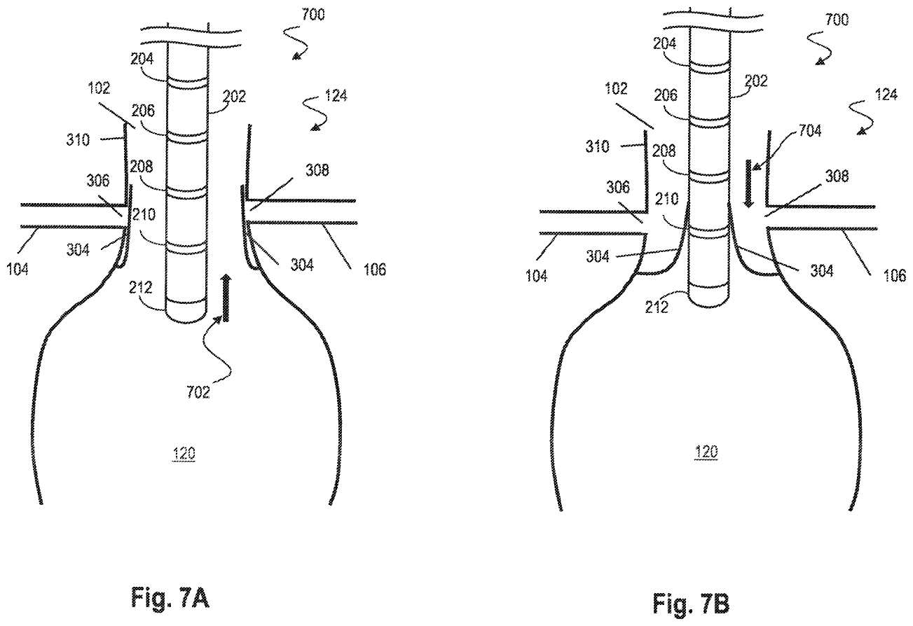

The static pressure measurements received by the processing system may include the static pressure measurements obtained by each of the plurality of pressure sensors of the elongate member 202. For example, the static pressure measurements received by the processing system may include a first static pressure measurement obtained by a first pressure sensor (see, e.g., sensor 210 of FIGS. 7A and 7B) positioned on a first side of the valve and a second static pressure measurement obtained by a second pressure sensor (see, e.g., sensor 208 of FIGS. 7A and 7B) positioned on a second side of the valve opposite the first side of the valve. The processing system may receive static pressure measurements from the plurality of pressure sensors throughout the cardiac cycle of the patient, including during systole and/or diastole. In some aspects, the processing system may determine a pressure differential between the first static pressure measurement and the second static pressure measurement at different points in time during the cardiac cycle of the patient. In this regard, the heartbeat of the patient may be paced by the elongate member 202 (e.g., using electrode(s) 212) while the static pressure measurements are obtained by the plurality of pressure sensors.

In some instances, the processing system may identify the pressure sensor(s) on each side of a valve based on the static pressure measurements received from the plurality of pressure sensors. That is, the processing system may be configured to determine which of the plurality of pressure sensors are the first pressure sensor and the second pressure sensor based on the signals received from the plurality of pressure sensors indicative of the static pressure measurements obtained by the plurality of pressure sensors. In this regard, pressure sensors on the same side of the valve typically have similar static pressure measurements throughout the heartbeat cycle of the patient, whereas pressure sensors on opposing sides of the valve will have notable variations in the associated static pressure measurements associated with the valve opening in closing during the heartbeat cycle. In some instances, the processing system may utilize measurements from the pressure sensors closest to the valve for additional processing. In some instances, the processing system may utilize measurements from at least one pressure sensor that is not the closest to the valve (e.g., second, third, or fourth closest on a side of the valve) for additional processing. For example, in situations where a pressure sensor is located very close to the transition between sides of the valve, the processing system may utilize a different pressure sensor in an effort to avoid unwanted abnormalities in the pressure data. In this regard, the pressure sensors further from the valve (on either side) may provide a better representation of the pressure in the chamber than those closer to the valve. For example, the turbulent flow of the blood through the valve can affect the pressure readings, sometimes more so on the outbound side of the valve than the inbound side. Accordingly, in some aspects the processing system may identify the pressure sensors closest to the valve and/or within a certain distance of the valve (e.g., based on position signals and/or pressure signals) and exclude measurements from those pressure sensors from other calculations and/or processing performed by the processing system (or associated components).

The processing system may determine a pressure differential between the first static pressure measurement and the second static pressure measurement. The processing system may determine the pressure differential between the first static pressure measurement and the second static pressure measurement at different points in time during a cardiac cycle of the patient. In this regard, the heartbeat of the patient may be paced by the elongate member 202 (e.g., using electrode(s) 212) while the static pressure measurements are obtained by the plurality of pressure sensors. The processing system may be configured to control at least one electrode of the elongate member 202 to pace the heartbeat of the patient. The processing system may determine a maximum pressure differential between the static pressure measurements obtained by the first pressure sensor and the static pressure measurements obtained by the second pressure sensor during a heartbeat cycle and/or an average of the maximum pressure differential over a plurality of heartbeat cycles. The pressure difference between the measurements of the first and second pressure sensors may be used as the “gradient” across the valve. In this regard, the gradient can provide an indication of how well sized a replacement valve is and/or if there are any issues with leaks or regurgitation of the replacement valve. The gradient can be used in a diagnostic sense (e.g., to evaluate a natural or previous replacement valve) or as a measure of success of an implant procedure.

The processing system may output to a display in communication with the processing system an indication of the pressure differential between the first static pressure measurement and the second static pressure measurement. In this regard, the indication of the pressure differential may include a numerical value representative of a difference between the first static pressure measurement and the second static pressure measurement, which may be the determined pressure differential in some instances, and/or the first static pressure measurement and the second static pressure measurement (e.g., the associated numerical values). In some instances, the indication of the pressure differential may be color coded (e.g., green for an acceptable value, yellow for questionable value, and red for an unacceptable value).

In some aspects, the processing system may receive positioning signals from the plurality of pressure sensors. The positioning signals may be based on ultrasound positioning signals transmitted by an ultrasound imaging device. The ultrasound imaging device may include an external ultrasound imaging system (e.g., an external ultrasound probe, a transthoracic echocardiography (TTE) system, etc.) and/or an internal ultrasound imaging system (e.g., transesophageal echocardiography (TEE) system, or an intracardiac echocardiography (ICE) system, and/or intravascular ultrasound (IVUS) system). In this regard, the processing system may track the position and/or orientation of the distal portion of the elongate member 202 using piezoelectric pressure sensors (e.g., sensors 204, 206, 208, and/or 210 in some instances). In this regard, the piezoelectric pressure sensors may receive and/or detect the ultrasound positioning signals/beams transmitted by the ultrasound imaging device and send electrical signals representative of the detected beams to the processing system. In some aspects, the ultrasound positioning signals/beams cause an electrical signal due to the piezoelectric nature of the pressure sensors. In some instances, ultrasound transducers may be positioned within the distal portion of the elongate member in addition to and/or in lieu of the piezoelectric pressure sensors and provide similar positioning signals to the processing system in response to the ultrasound positioning signals transmitted by the ultrasound imaging device.

The processing system may determine the location of the various piezoelectric pressure sensors (and/or ultrasound transducers) relative to the field of view of the ultrasound imaging device and associated patient anatomy based on the time-of-flight between the transducer and/or transducer array of the ultrasound imaging device and the piezoelectric pressure sensors (and/or ultrasound transducers). The time-of-flight may be determined using the relative timing between the trigger signals for the ultrasound positioning signals and the signals from the piezoelectric pressure sensors (and/or ultrasound transducers) based on the detected/received ultrasound positioning signals. Based on the received positioning signals, the processing system may determine the positions and/or orientations of the plurality of pressure sensors within the patient.

Because the piezoelectric pressure sensors (and/or ultrasound transducers) may be exposed to internal and external noise, the processing system may determine which portion of the signal received from the piezoelectric pressure sensors (and/or ultrasound transducers) is representative of the ultrasound positioning signal transmitted by the ultrasound imaging device. In some aspects, the piezoelectric pressure sensors are utilized for both static pressure measurements and positioning. When a piezoelectric sensor is insonified by ultrasound waves (e.g., the ultrasound positioning signals transmitted by the ultrasound imaging device), a current is generated, which can be synchronized with the ultrasound firing pattern to identify the position of the sensor within the ultrasound field of view. In this regard, the signal generated by the ultrasound energy interacting with the piezoelectric pressure sensors is at a higher frequency as compared to the signal generated by piezoelectric pressure sensor based on the cardiac or vascular static pressure. Accordingly, in some instances the processing system may be configured to filter the signals received from the plurality of pressure sensors indicative of the static pressure measurements from the positioning signals received from the plurality of pressure sensors.

In some aspects, the electrical signals from each piezoelectric pressure sensor may pass through a splitter, where one output arm goes to an amplifier, a high-pass filter, then to an analog to digital converter (ADC) and to the processing system for processing the position information. The other output arm of the splitter may go through a low pass filter (and any additional signal conditioning and/or filtering hardware), then an ADC (the same or a different ADC as used by the other output arm), and then to the processing system for processing the static pressure information. Alternatively, the signal from each piezoelectric pressure sensor may follow a single signal path and the position and pressure information can be separated in signal processing software using digital high-pass and/or low-pass filtering.

The processing system may output to an imaging display an indication of a position of the distal portion of the elongate member within the patient based on the determined locations of the plurality of pressure sensors. For example, in some instances the indication of the position of the distal portion of the multiple sensor device within the patient includes an overlay on an ultrasound image of anatomy of the patient based on ultrasound data obtained from a TEE device, a TTE device, an ICE device, and/or other ultrasound imaging device. In some aspects, the processing system may be further configured to output to the imaging display an indication of an orientation of the distal portion of the multiple sensor device within the patient based on the determined locations of the plurality of pressure sensors. In some instances, the position of each sensor may be rendered as an overlay, underlay, or otherwise on an ultrasound image associated with the ultrasound imaging device and/or on any other co-registered imaging modality (i.e. fluoroscopy, CT, or other anatomical model). Further, the static pressure measurement for each sensor location, as well as information derived therefrom (e.g., pressure differentials and indications thereof) can be displayed on the same or a different display and/or user interface.

The sensors and/or the electrode(s) of the elongate member 202 may be spaced from one another by one or more known, fixed distances. For example, each of the sensors may be spaced apart from the other adjacent sensors by a fixed distance along the length of the elongate member. Similarly, with a plurality of electrodes, each of the electrodes may be spaced apart from the other adjacent sensors by a fixed distance along the length of the elongate member. When a single electrode is utilized (as shown in FIG. 2), the electrode 212 may be spaced from the distal most sensor (e.g., sensor 210) by a fixed distance. In some instances, the fixed distance between the electrode 212 and the distal most sensor may be based on the anatomy the elongate member 202 is intended to be used in. For example, the electrode 212 may be spaced from the sensor 210 by a distance that allows the electrode 212 to be in contact with or in close proximity to a tissue wall of a chamber of the heart for pacing while the sensor is positioned in proximity to valve connected to the chamber. The relative positions of the sensors and/or electrodes of the elongate member 202 may be utilized to track the position and/or orientation of the distal portion of the elongate member 202 within the patient anatomy as described above and herein.

In some instances, the plurality of sensors of the elongate member may be used to locate structures (e.g., valves, chambers of the heart, etc.) and/or abnormalities within the patient anatomy (e.g., blockages, bifurcations, etc.), including some features that may not be visible and/or detectable using external imaging. In some instances, a series of pressure measurements is taken by the plurality of sensors of the elongate member 202 and the location of a valve relative to the plurality of sensors may be determined based on the pressure measurements received from the plurality of sensors. In this way, the elongate member 202 may provide pressure measurements on each side of the valve, which may be used for diagnosis and/or evaluation of the operation of a replacement valve, without the need to reposition the elongate member 202 and without exchanging devices.

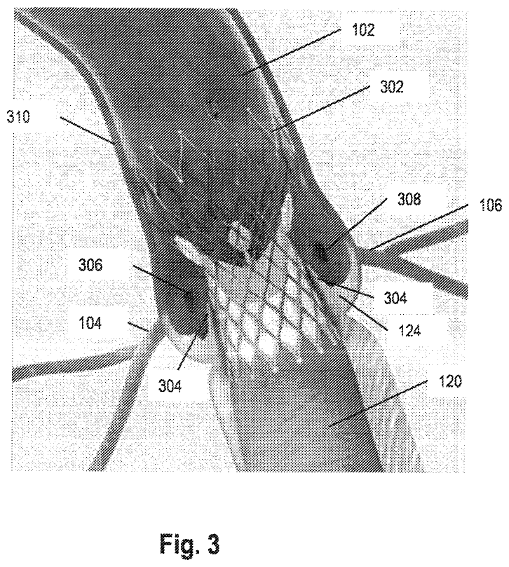

FIG. 3 is a cross-sectional view of an aortic valve replacement in a human heart according to aspects of the present disclosure. In some aspects, e.g., when aortic valve stenosis has occurred to the aortic valve 124 that keeps blood from flowing in the correct direction from the left ventricle 120 to the aorta 102, a transvenous/transcatheter aortic valve repair (TAVR) procedure may be performed to replace the aortic valve 124 (e.g., a natural aortic valve) with a replacement aortic valve 302. In some instances, portions of one or more leaflets 304 of the aortic valve 124 may be resected so that an opening 306 to the right coronary artery 104 and an opening 308 to the left main coronary artery 106 are formed. When the one or more leaflets 304 are pressed against the natural heart wall 310, the openings 306 and 308 remain unobstructed so that oxygen-rich blood may flow to the muscles of the human heart. The natural heart wall 310 may be a natural aorta wall, a natural heart chamber wall, or a natural aortic valve wall. In accordance with the present disclosure, the elongate member 202 may be utilized as part of the aortic valve replacement procedure, including without limitation to diagnose the need for an aortic valve replacement and/or evaluate the efficacy of an aortic valve replacement.

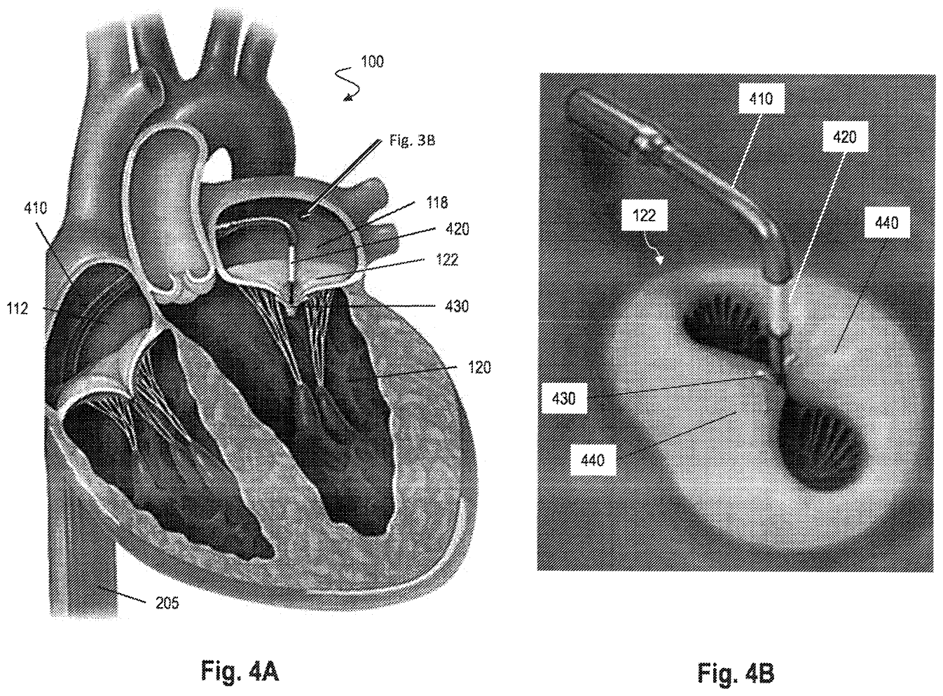

FIG. 4A is a cross-sectional view of a human heart 100 undergoing a mitral valve transcatheter edge-to-edge repair (TEER) procedure, according to aspects of the present disclosure. Visible are the left atrium 118, left ventricle 120, and mitral valve 122. A deployment catheter 410 has entered the human heart 100 through the inferior vena cava 205, through the right atrium 112, across the interatrial septum (transeptal access), and into the left atrium 118. A deployment device 420 has emerged from the deployment catheter 410 to deploy a mitral valve clip 430. The mitral valve clip 430 may hold together leaflets of the mitral valve 122.

FIG. 4B is a close-up view of the TEER procedure of FIG. 4A according to aspects of the present disclosure. Visible are the deployment catheter 410, the deployment device 420, and the mitral valve clip 430. The mitral valve clip 430 holds together leaflets 440 of the mitral valve 122 to treat/reduce/prevent mitral valve regurgitation. In accordance with the present disclosure, the elongate member 202 may be utilized as part of the TEER procedure, including without limitation to diagnose the need for mitral valve repair and/or evaluate the efficacy of the mitral valve repair.

The aortic valve replacement of FIG. 3 and the mitral valve TEER procedure of FIGS. 4A and 4B are shown here for exemplary purposes only. It is understood that other heart valves and heart valve repair/replacement procedure types may benefit from the use of the elongate member 202 and thus fall within the scope of the present disclosure. The technology described herein, including the elongate member 202 and associated systems and methods, may be applied to any heart prosthesis (e.g., repair device, replacement device), in or between any heart chambers, where it may be desirable to obtain multiple pressure measurements (or other measurements) at different locations while pacing the heartbeat of the patient and/or utilize the sensors to determine the position and/or orientation of the elongate member within the patient anatomy. Further, the technology described herein, including the elongate member 202 and associated systems and methods, may be applied to any suitable location (e.g., aorta, inferior vena cava (IVC), superior vena cava (SVC), pulmonary arteries/veins, heart chamber, such as left atrium, right atrium, left ventricle, right ventricle, left atrial appendage, etc.) and/or tissue (e.g., valve, such as tricuspid valve, pulmonary valve, mitral valve, aortic valve, etc.), including non-cardiac applications.

FIG. 5 is schematic diagram of a system 500 according to aspects of the present disclosure. The system 500 may be configured to evaluate (e.g., diagnose, assess, monitor), display, and/or control (e.g., modify) one or more aspects of a cardiac valve replacement or other medical procedure. In this regard, the system 500 may be used in the context of structural heart procedures, including those involving cardiac valves, coronary vessels, and/or heart tissue (e.g., the myocardium). As illustrated, the system 500 may include a processing system 510 in communication with one or more display device(s) 520 (e.g., an electronic display or monitor, etc.), an input device 530 (e.g., a user input device, such as a keyboard, mouse, joystick, microphone, and/or other controller or input device, etc.), a cutting/ablation subsystem 540, a balloon subsystem 550, one or more imaging device(s) 560 (e.g., external x-ray, angiography, fluoroscopy, ultrasound, ICE, TEE, TEE, IVUS, OCT, etc.), and/or a multiple sensor device 570 (e.g., elongate member 202). As illustrated, the system 500 may further include a delivery and/or treatment catheter 580 (e.g., a SAVR delivery catheter, a TAVR delivery catheter, a TEER catheter, etc.) and a valve 590 (e.g., a SAVR valve, a TAVR valve, etc.) or other treatment device and/or implantable device.

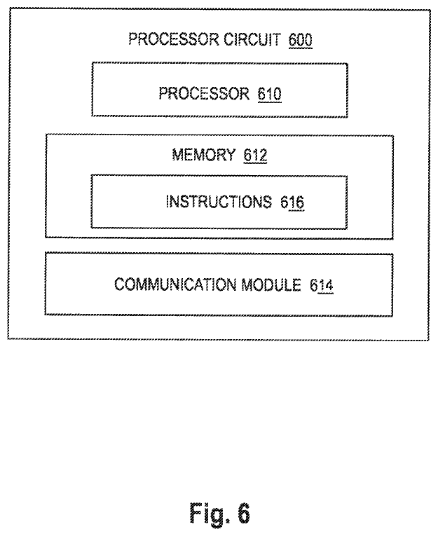

The processing system 510 is generally representative of any device suitable for performing the processing, analysis, and/or control techniques disclosed herein. In some aspects, the processing system 510 includes a processor circuit, such as the processor circuit 600 of FIG. 6. In some aspects, the processing system 510 is programmed to execute steps associated with the data acquisition, analysis, and/or instrument (e.g., device) control described herein. Accordingly, it is understood that any steps related to data acquisition, data processing, instrument control, and/or other processing or control aspects of the present disclosure may be implemented by the processing system 510 (e.g., computing device) using corresponding instructions stored on or in a non-transitory computer readable medium accessible by the computing device. In some instances, the processing system 510 is a console device. Further, it is understood that in some instances the processing system 510 includes one or a plurality of computing devices, such as computers, with one or a plurality of processor circuits. In this regard, it is understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

FIG. 6 is a schematic diagram of a processing system according to aspects of the present disclosure. The processor circuit 600 may be implemented in and/or as part of the processing system 510 of FIG. 5. As shown, the processor circuit 600 may include a processor 610, a memory 612, and a communication module 614. These elements may be in direct or indirect communication with each other, for example via one or more buses.

The processor 610 may include a central processing unit (CPU), a digital signal processor (DSP), an ASIC, a controller, an FPGA, another hardware device, a firmware device, or any combination thereof configured to perform the operations described herein. The processor 610 may also be implemented as a combination of computing devices, e.g., a combination of a DSP and a microprocessor, a plurality of microprocessors, one or more microprocessors in conjunction with a DSP core, or any other such configuration.

The memory 612 may include a cache memory (e.g., a cache memory of the processor 610), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In some instances, the memory 612 includes a non-transitory computer-readable medium. The memory 612 may store instructions 616. The instructions 616 may include instructions that, when executed by the processor 610, cause the processor 610 to perform the operations described herein with reference to the processing system 510 (FIG. 5). Instructions 616 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 614 may include any electronic circuitry, logic circuitry, and/or optical pathways and components to facilitate direct or indirect communication of data between various components of the processor circuit 600 and/or the processing system 510 (FIG. 5). Additionally, or alternatively, the communication module 614 may facilitate communication of data between the processor circuit 600, the display device(s) 520, the input device 530, the cutting/ablation subsystem 540, the balloon subsystem 550, the imaging device(s) 560, the multiple sensor device 570, the delivery/treatment catheter 580, and/or the like. In this regard, the communication module 614 may be an input/output (I/O) device interface, which may facilitate communicative coupling between the processor circuit 600 and (I/O) devices, such as the input device 530. Moreover, the communication module 614 may facilitate wireless and/or wired communication between various elements of the processor circuit 600 and/or the devices and systems of the system 500 using any suitable communication technology, such as a cable interface such as a USB, micro-USB, Lightning, or Fire Wire interface, Bluetooth, Wi-Fi, ZigBee, Li-Fi, or cellular data connections such as 2G/GSM, 3G/UMTS, 4G/LTE/WiMax, or 5G.

Turning back now to FIG. 5, the imaging device(s) 560 may include an x-ray system, angiography system, fluoroscopy system, ultrasound system (including external ultrasound imaging systems (e.g., ultrasound probes and/or TTE) as well as internal ultrasound imaging systems (e.g., ICE, TEE, and/or IVUS systems), computed tomography (CT) system, a magnetic resonance imaging (MRI) system, an OCT system, other suitable imaging devices, and/or combinations thereof. The imaging device(s) 560 may additionally or alternatively include a nuclear medicine imaging device, such as a gamma camera or a single-photon emission computed tomography (SPECT) system, other suitable devices, and/or combinations thereof. In some aspects, the imaging device(s) 560 may be configured to acquire imaging data of anatomy, such as the heart and blood vessels, while the imaging device(s) 560 is positioned outside of the body of the patient. The imaging data may be visualized in the form of two-dimensional and/or three-dimensional images of the heart, blood vessel, and/or other anatomy. In some aspects, the imaging device(s) 560 may include an internal device that is positioned inside the body of the patient. For example, the imaging device(s) 560 may include an intracardiac echocardiography (ICE) catheter that obtains images while positioned within a heart chamber. In some aspects, the imaging device(s) 560 may be positioned outside of the particular anatomy that is being imaged (e.g., blood vessels and/or heart), but is positioned inside the patient body. For example, the imaging device(s) 560 may include a transesophageal echocardiography (TEE) probe that obtains images while positioned within an esophagus.

Moreover, the imaging device(s) 560 may obtain images of the heart that are indicative of the health of the cardiac muscle or myocardium. In particular, the imaging device(s) 560 may be configured to acquire imaging data that illustrates myocardial perfusion (e.g., myocardial perfusion imaging (MPI) data). For example, MPI data may be collected by imaging a radiopharmaceutical agent, such as thallium, in the patient's heart muscle using a SPECT system. The imaging data may illustrate vasculature and/or muscle mass with blood flow and/or vasculature and/or muscle mass that lack of blood flow in areas of the heart.

Additionally, or alternatively, the imaging device(s) 560 may be utilized to track the location of an intraluminal device within the patient, including the position and/or orientation of a distal portion of the intraluminal device with the anatomy of the patient. For example, in some aspects, the processing system may receive positioning signals from a plurality of pressure sensors of the intraluminal device. The positioning signals may be based on ultrasound positioning signals transmitted by an ultrasound imaging device. The ultrasound imaging device may include an external ultrasound imaging system (e.g., external ultrasound probe, a transthoracic echocardiography (TTE) system, etc.) and/or an internal ultrasound imaging system (e.g., transesophageal echocardiography (TEE) system, or an intracardiac echocardiography (ICE) system, and/or intravascular ultrasound (IVUS) system). In this regard, the processing system may track the position and/or orientation of the distal portion of the intraluminal device using piezoelectric pressure sensors. In this regard, the piezoelectric pressure sensors may receive and/or detect the ultrasound positioning signals/beams transmitted by the ultrasound imaging device and send electrical signals representative of the detected beams to the processing system. In some aspects, the ultrasound positioning signals/beams cause an electrical signal to be generated by the pressure sensors due to the piezoelectric nature of the pressure sensors. In some instances, ultrasound transducers may be positioned within the distal portion of the intraluminal device in addition to and/or in lieu of the piezoelectric pressure sensors and provide similar positioning signals to the processing system in response to the ultrasound positioning signals transmitted by the ultrasound imaging device.

The processing system may determine the location of the various piezoelectric pressure sensors (and/or ultrasound transducers) relative to the field of view of the ultrasound imaging device and associated patient anatomy based on the time-of-flight between the transducer and/or the transducer array of the ultrasound imaging device and the piezoelectric pressure sensors (and/or ultrasound transducers). The time-of-flight may be determined using the relative timing between the trigger signals for the ultrasound positioning signals and the signals from the piezoelectric pressure sensors (and/or ultrasound transducers) based on the detected/received ultrasound positioning signals. Based on the received positioning signals, the processing system may determine the positions and/or orientations of the plurality of pressure sensors within the patient.

As a further example, the imaging data obtained by the imaging device(s) may include imaging data representative of one or more radiopaque markers embedded in an intraluminal device such as, for example, a cutting/ablation device 546 of the cutting/ablation subsystem 540, a balloon device 554 of the balloon subsystem 550, the multiple sensor device 570, and/or the delivery/treatment catheter 580. Imaging the radiopaque markers may enable tracking of the intraluminal device, including the position and/or orientation of a distal portion of the intraluminal device with the anatomy of the patient. In this regard, the position and/or orientation of the intraluminal device may be determined by the processing system 510 based on the signals received from the sensors of the intraluminal device and/or the relative locations of the radiopaque markers. An indication of the determined position and/or orientation of the intraluminal device may be overlayed, underlaid, or otherwise included on images of the patient anatomy displayed on the display device(s) 520. In this regard, the position and/or orientation of the intraluminal device may be co-registered across multiple different imaging and/or data types and a corresponding indication included for each of the different imaging and/or data types.

The balloon subsystem 550 may control a pump 552 to capture leaflets of a degenerated natural aortic valve or a degenerated replacement aortic valve. In some aspects, the human heart/heart cycle has two portions: systole and diastole. In that regard, the balloon device 554 may be advanced through the aortic valve during systole when the heart muscle contracts and pumps blood from the chambers into the arteries. Then, during diastole when the heart muscle relaxes and the chambers of the heart are allowed to fill with blood, the leaflets of the aortic valve may press against the outer surface of the balloon device 554. The pump 552 of the balloon subsystem 550 may be configured to selectively expand and contract a balloon of the balloon device 554. In this regard, pump 552 may be configured to transition the balloon between an expanded state and an unexpanded state.

The cutting/ablation subsystem 540 may control a laser source 542 and/or motor 544 for controlling a laser cutter, ablation device, and/or cutting tip disposed on a distal end or portion of a cutting/ablation device 546. For example, in some instances once the leaflets of the degenerated natural aortic valve or a degenerated replacement aortic valve are located, grasped, and/or secured in place, the cutting/ablation subsystem 540 may cut, ablate, or otherwise remove portions (or all) of the leaflets. In some instances, the cutting/ablation subsystem 540 may be configured to activate the laser source 542 such that one or more optical fibers disposed on a distal end of the cutting/ablation device 546 may cut and/or resect the degenerated leaflets of the natural aortic valve or the replacement aortic valve. In some aspects, the cutting/ablation subsystem 540 may be configured to activate the motor 544 such that one or more mechanical blades (or other cutting mechanisms) disposed on a distal end of the cutting/ablation device 546 may cut and/or resect the degenerated leaflets of the natural aortic valve or the replacement aortic valve. Once the leaflets of the degenerated natural aortic valve or a degenerated replacement aortic valve have been removed, the delivery/treatment catheter 580 may deliver the valve 590 (or other implant or treatment device) to the valve area of the human heart, such that the valve 590 replaces either the degenerated natural aortic valve or a degenerated replacement aortic valve.

The display device(s) 520 may be communicatively coupled to the processing system 510. In some aspects, the display device(s) 520 may be a component of the processing system 510, while in other aspects, the display device(s) 520 may be distinct from the processing system 510. In some aspects, the display device(s) 520 may include a monitor integrated into a console device or a standalone monitor (e.g., a flat panel or flat screen monitor). The processing system 510 may be configured to generate one or more visual displays (e.g., screen displays) based on imaging data from the imaging device(s) 560. The processing system 510 may provide (e.g., output) the screen display to the display device(s) 520. For example, the display device(s) 520 may be configured to output (e.g., display) a two-dimensional image and/or a two-dimensional representation of the heart, blood vessels, and/or other anatomy, which may be included in the screen display. In some aspects, the display device(s) 520 may be configured to output a three-dimensional graphical representation of the heart, blood vessels, and/or other anatomy. For instance, the display device(s) 520 may be a holographic display device configured to output a three-dimensional holographic display of anatomy. Any suitable display device(s) may be utilized within the scope of this disclosure, including self-contained monitors, projection/screen systems, head-up display systems, etc. The display device(s) 520 may implement principles based on moving reflective microelectromechanical systems (MEMS), laser plasma, electro-holography, etc. In some aspects, the display device(s) 520 may be implemented as a bedside controller having a touch-screen display.

The input device(s) 530 may be communicatively coupled to the processing system 510. The input device(s) 530 may be a peripheral device, such as a touch sensitive pad, a touchscreen, a joystick, a keyboard, mouse, trackball, a microphone, an imaging device, and/or the like. In other aspects, the user interface device is part of the display device(s) 520, which may be a touch-screen display, for example. Moreover, a user may provide an input to the processing system 510 via the input device(s) 530. In particular, the input device(s) 530 may enable a user to control, via inputs to the processing system 510, one or more of the components of the system 500, such as the cutting/ablation subsystem 540, the balloon subsystem 550, the imaging device(s) 560, the multiple sensor device 570, the delivery/treatment catheter 580, and/or the processing system 510 itself. Additionally, or alternatively, the input device(s) 530 may facilitate interaction with a screen display provided at the display device(s) 520. For instance, a user may select, edit, view, or interact with portions of the screen display (e.g., a GUI) provided at the display device(s) 520 via the input device 530.

The system 500 may include various connectors, cables, interfaces, connections, etc., to communicate between the elements of the cutting/ablation subsystem 540, the balloon subsystem 550, the imaging device(s) 560, the multiple sensor device 570, the delivery/treatment catheter 580, the processing system 510, the display device(s) 520, and/or the input device(s) 530. In some aspects, for example, the communication module 614 (FIG. 6), which may be included in the processing system 510, may include connectors, interfaces, and/or the like. In this regard, the processing system 510 may communicate and/or control one or more components of the processing system 510 via mechanical, electromechanical, wireless, and/or optical signaling and/or controls. Further, the illustrated communication pathways are exemplary in nature and should not be considered limiting in any way. In this regard, it is understood that any communication pathway between the components of system 500 may be utilized, including physical connections (including electrical, optical, and/or fluid connections), wireless connections, and/or combinations thereof. In this regard, it is understood that one or more of the components of the system 500 may communicate via a wireless connection in some instances. In some instances, one or more components of the system 500 and/or other systems (e.g., of a hospital or health services provider) communicate via a communication link over a network (e.g., intranet, internet, telecommunications network, and/or other network).

Referring now to FIGS. 7A and 7B, shown therein is the use of a multiple sensor device (e.g., elongate member 202) in the context of an aortic valve procedure 700 in accordance with aspects of the present disclosure. In this regard, FIG. 7A is a diagrammatic cross-sectional view of a distal portion of a multiple sensor device positioned across an aortic valve in an open state (e.g., during systole) according to aspects of the present disclosure. FIG. 7B is a diagrammatic cross-sectional view of a distal portion of a multiple sensor device positioned across an aortic valve in a closed state (e.g., during diastole) according to aspects of the present disclosure. Visible are the natural heart wall 310, the aortic valve leaflets 304, the right coronary artery 104, and the left main coronary artery 106, including openings 306 and 308 to the right and left coronary arteries, respectively. In the aspects of FIG. 7A, during systole, the aortic valve leaflets 304 open and allow blood to flow from the left ventricle 120 to the aorta 102 as indicated by directional arrow 702. During systole, the distal portion of the elongate member 202 may be advanced between the aortic valve leaflets 304 such that the elongate member 202 extends at least partially through the aortic valve. In this regard, one or more of the sensors (e.g., sensor 210) and/or one or more of the electrodes (e.g., electrode 212) may be positioned on the left ventricle side of the aortic valve while one or more sensors (e.g., sensors 204, 206, and 208) and/or one or more of the electrodes may be positioned on the aorta side of the aortic valve. As shown in FIG. 12B, during diastole, the aortic valve leaflets 304 may be pushed by the flow of blood from the aorta 102 toward the left ventricle 120, as indicated by directional arrow 704, such that the aortic valve leaflets 304 rest against and/or contact the elongate member.

In some aspects, the processing system may receive signals indicative of static pressure measurements obtained by the plurality of pressure sensors (i.e., sensors 204, 206, 208, and/or 210) of the elongate member 202 while positioned across the aortic valve. In this regard, the static pressure measurements received by the processing system may include the static pressure measurements obtained by each of the plurality of pressure sensors of the elongate member 202. For example, the static pressure measurements received by the processing system may include a first static pressure measurement obtained by a first pressure sensor (see, e.g., sensor 210 of FIGS. 7A and 7B) positioned on a first side of the valve and a second static pressure measurement obtained by a second pressure sensor (see, e.g., sensor 208 of FIGS. 7A and 7B) positioned on a second side of the valve opposite the first side of the valve. The processing system may receive static pressure measurements from the plurality of pressure sensors throughout the cardiac cycle of the patient, including during systole and/or diastole. In some aspects, the processing system may determine a pressure differential between the first static pressure measurement and the second static pressure measurement at different points in time during the cardiac cycle of the patient.

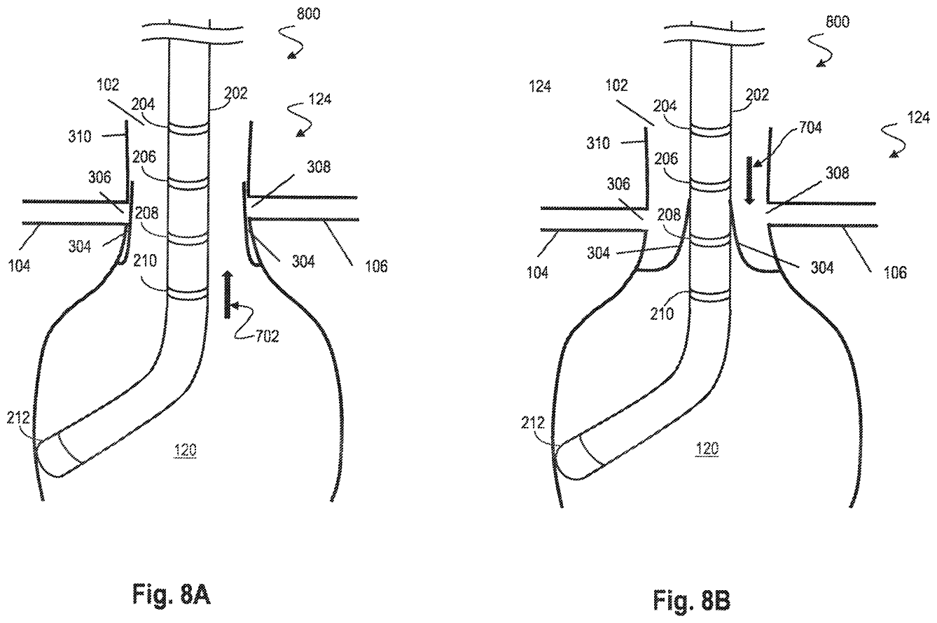

Referring now to FIGS. 8A and 8B, shown therein is the use of a multiple sensor device (e.g., elongate member 202) in the context of an aortic valve procedure 800 in accordance with aspects of the present disclosure. FIGS. 8A and 8B are similar in many respects to FIGS. 7A and 7B. For sake of brevity, the description of common or similar features will not be repeated, though the same reference numerals will be utilized in some instances. FIGS. 8A and 8B differ from FIGS. 7A and 7B in that they show the electrode 212 of the elongate member 202 in contact with (or in close proximity to) a tissue wall of the left ventricle 120. In this regard, the electrode 212 may be utilized by the processing system to pace the heartbeat of the patient, including while static pressure measurements are obtained by the plurality of pressure sensors (e.g., sensors 204, 206, 208, and/or 210) of the elongate member 202.

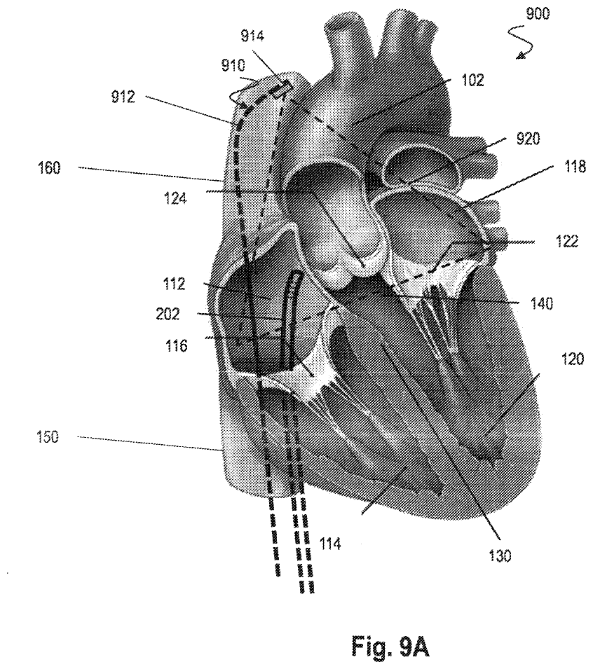

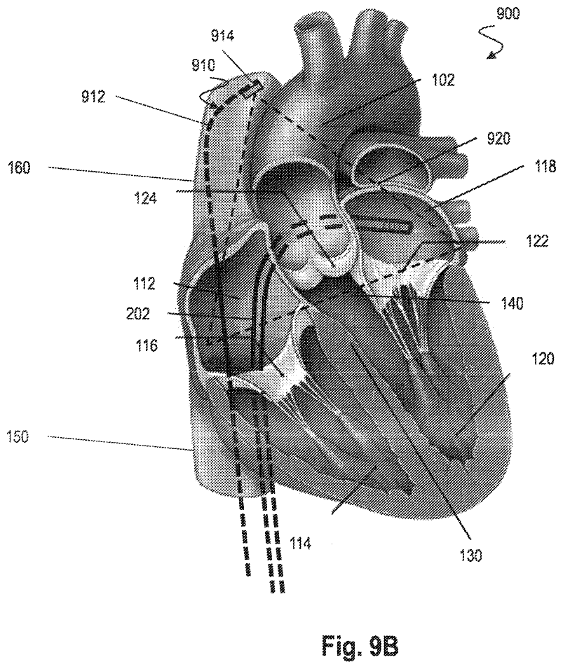

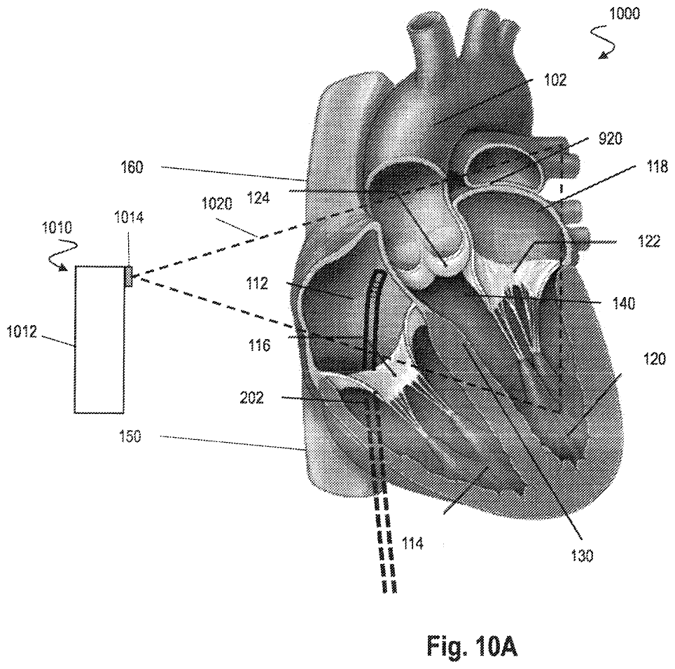

Referring now to FIGS. 9A and 9B, shown therein is the use of a multiple sensor device (e.g., elongate member 202) in the context of a structural heart procedure 900 in accordance with aspects of the present disclosure. FIG. 9A is a cross-sectional view of a human heart being imaged by an intracardiac echography (ICE) catheter 910 for tracking positioning of a distal portion of a multiple sensor device, according to aspects of the present disclosure. FIG. 9B is a cross-sectional view of the human heart being imaged by the ICE catheter 910 for tracking positioning of the distal portion of the multiple sensor device similar to that of FIG. 9A but showing the distal portion of the multiple sensor device in a different location, according to aspects of the present disclosure.

Referring to FIGS. 9A and 9B, visible are the right atrium 112, right ventricle 114, tricuspid valve 116, left atrium 118, left ventricle 120, mitral valve 122, aorta 102, aortic valve 124, left ventricular outflow tract 140, septum 130, inferior vena cava (IVC) 150, and superior vena cava (SVC) 160. The ICE catheter 910 may include a flexible elongate member 912 and an ultrasound transducer array 914. In the example shown, the ICE catheter 910 is positioned such that the field of view 920 of the ultrasound transducer array 914 has a view of both the aortic valve 124 and the mitral valve 122. The field of view 920 of the ultrasound transducer array 914 can be selected based on the position and/or orientation (e.g., deflection) of the distal portion of the ICE catheter 910. Instead of or in addition, the field of view 920 of the transducer array 914 can be selected using beam steering. The ICE catheter 910 may travel through the IVC 150, through the right atrium and into the SVC 160, for imaging of the aortic valve 124 and mitral valve 122 via the SVC 160. In this position and orientation, the ICE catheter 910 can image the aortic valve 124 and the mitral valve 122 simultaneously and, therefore, track the positioning of the distal portion of the elongate member 202 for use in both aortic valve and/or mitral valve procedures. In other aspects, the ICE catheter 910 can image the mitral valve 122 and/or the aortic valve 124 transeptally (e.g., while positioned inside the left atrium).

As shown in FIGS. 9A and 9B, the elongate member 202 may be advanced through the patient anatomy. For example, FIG. 9A shows the distal portion of the elongate member 202 within the right atrium 112, while FIG. 9B shows the distal portion of the elongate member 202 within the left atrium 118. The position and/or orientation of the distal portion of the elongate member 202 may be tracked by a processing system (e.g., processing system 510) in communication with the elongate member 202 and/or the ICE catheter 910. As discussed previously, the processing system may receive positioning signals from the plurality of sensors of the elongate member. The positioning signals may be based on ultrasound positioning signals transmitted by the ICE catheter 910. In this regard, the processing system may track the position and/or orientation of the distal portion of the elongate member 202 based on signals received from piezoelectric pressure sensors (e.g., sensors 204, 206, 208, and/or 210 in some instances). In this regard, the piezoelectric pressure sensors may receive and/or detect the ultrasound positioning signals/beams transmitted by the ICE catheter 910 and send electrical signals representative of the detected beams to the processing system. In this regard, the processing system may determine the location and/or orientation of each of the sensors of the elongate member 202 within the field of view 920 of the ICE catheter 910 based on the time-of-flight between the transducer and/or transducer array of the ICE catheter 910 and the sensors of the elongate member 202. The time-of-flight may be determined using the relative timing between the trigger signals for the ultrasound positioning signals and the signals received from the sensors of the elongate member 202 associated with the detected/received ultrasound positioning signals.

Based on the received positioning signals from the sensors of the elongate member 202, the processing system may determine the positions and/or orientations of the plurality of pressure sensors within the patient. For example, the processing system may determine that the distal portion of the elongate member 202 is positioned within right atrium 112 (as shown in FIG. 9A), positioned within the left atrium 118 (as shown in FIG. 9B), and/or otherwise positioned within a portion of the heart and/or associated vasculature. The processing system may output to an imaging display an indication of a position of the distal portion of the elongate member 202 within the patient based on the determined locations of the plurality of sensors. For example, in some instances the indication of the position of the distal portion of the multiple sensor device within the patient includes an overlay on an ultrasound image of anatomy of the patient generated based on ultrasound data from the ICE catheter 910.

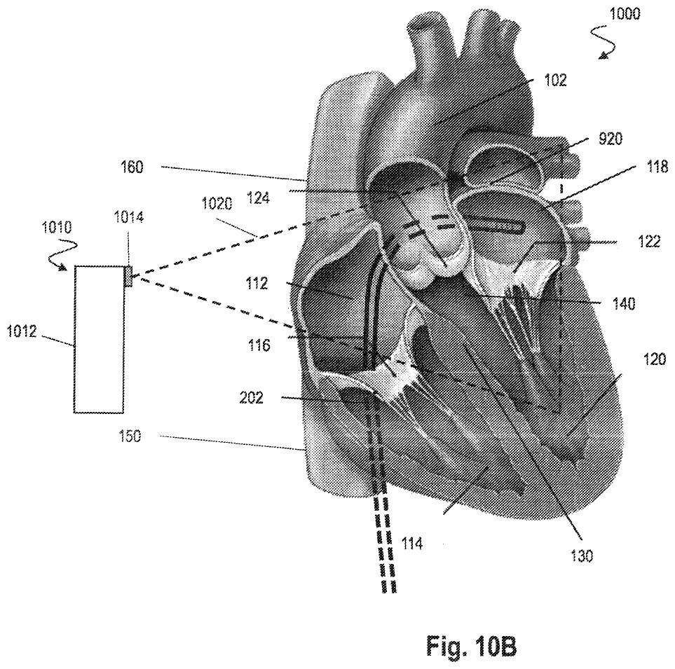

Referring now to FIGS. 10A and 10B, shown therein is the use of a multiple sensor device (e.g., elongate member 202) in the context of a structural heart procedure 1000 in accordance with aspects of the present disclosure. FIG. 10A is a cross-sectional view of a human heart being imaged by an ultrasound device 1010 for tracking positioning of a distal portion of a multiple sensor device, according to aspects of the present disclosure. FIG. 10B is a cross-sectional view of the human heart being imaged by the ultrasound device 1010 for tracking positioning of the distal portion of the multiple sensor device similar to that of FIG. 10A but showing the distal portion of the multiple sensor device in a different location, according to aspects of the present disclosure. FIGS. 10A and 10B are similar in many respects to FIGS. 9A and 9B. For sake of brevity, the description of common or similar features will not be repeated, though the same reference numerals will be utilized in some instances. FIGS. 10A and 10B differ from FIGS. 9A and 9B in that they show a different type of ultrasound device being utilized. In this regard, whereas FIGS. 9A and 9B showed the use of an ICE catheter, FIGS. 10A and 10B represent the use of ultrasound device 1010 positioned outside of the cardiac area of the patient, which may include use of an external ultrasound probe, a transthoracic echocardiography (TTE) probe, transesophageal echocardiography (TEE) probe, and/or otherwise. The ultrasound device 1010 may include a body 1012 and an ultrasound transducer array 1014.

The position and/or orientation of the distal portion of the elongate member 202 may be tracked by a processing system (e.g., processing system 510) in communication with the elongate member 202 and/or the ultrasound device 1010. As discussed previously, the processing system may receive positioning signals from the plurality of sensors of the elongate member. The positioning signals may be based on ultrasound positioning signals transmitted by the ultrasound device 1010. In this regard, the processing system may determine the location and/or orientation of each of the sensors of the elongate member 202 within a field of view 1020 of the ultrasound imaging device 1010 based on the time-of-flight between the ultrasound transducer array 1014 of the ultrasound imaging device 1010 and the sensors of the elongate member 202.

Based on the received positioning signals from the sensors of the elongate member 202, the processing system may determine the positions and/or orientations of the plurality of pressure sensors within the patient. For example, the processing system may determine that the distal portion of the elongate member 202 is positioned within right atrium 112 (as shown in FIG. 10A), positioned within the left atrium 118 (as shown in FIG. 10B), and/or otherwise positioned within a portion of the heart and/or associated vasculature. The processing system may output to an imaging display an indication of a position of the distal portion of the elongate member 202 within the patient based on the determined locations of the plurality of sensors. For example, in some instances the indication of the position of the distal portion of the multiple sensor device within the patient includes an overlay on an ultrasound image of anatomy of the patient generated based on ultrasound data from the ultrasound device 1010.

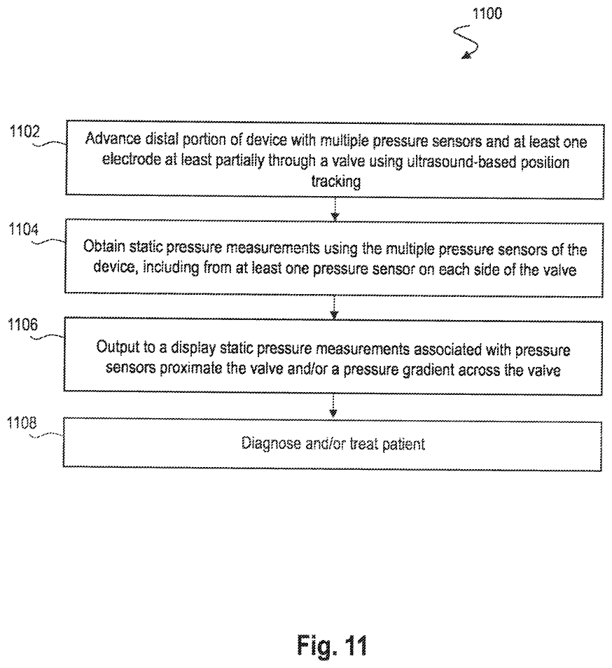

FIG. 11 is a flow diagram of a method 1100 of utilizing a multiple sensor device according to aspects of the present disclosure. It is understood that the actions of the method 1100 may be performed in a different order than shown in FIG. 11, additional actions can be provided before, during, and after the described actions, and/or some of the described actions can be replaced and/or eliminated. Some of the actions of the method 1100 can be carried out by one or more components and/or a user of the system 500. Further, the method 1100 can include any actions described above in the context of FIGS. 1-10.

At action 1102, the method 1100 may include advancing a distal portion of a device with multiple pressure sensors and at least one electrode at least partially through a valve using ultrasound-based position tracking. In this regard, action 1102 may include receiving, by a processing system from a multiple sensor device positioned within the patient, positioning signals from a plurality of pressure sensors spaced apart along a length of a distal portion of the multiple sensor device. The positioning signals may be based on ultrasound positioning signals transmitted by an ultrasound imaging device, such as an ultrasound imaging device associated with an external ultrasound imaging system (e.g., an external ultrasound probe, a transthoracic echocardiography (TTE) system, etc.) and/or an internal ultrasound imaging system (e.g., transesophageal echocardiography (TEE) system, or an intracardiac echocardiography (ICE) system, and/or intravascular ultrasound (IVUS) system).

Action 1102 may include determining the location of the various pressure sensors of the multiple sensor device relative to the field of view of the ultrasound imaging device. In some aspects, the locations may be determined based on the time-of-flight between the transducer and/or transducer array of the ultrasound imaging device and the pressure sensors. For example, the time-of-flight may be determined using the relative timing between the trigger signals for the ultrasound positioning signals and the signals from the pressure sensors based on the detected/received ultrasound positioning signals. Based on the received positioning signals, the positions and/or orientations of the plurality of pressure sensors within the patient may be determined.

Action 1102 may also include outputting, to an imaging display in communication with the processing system, an indication of a position of the distal portion of the multiple sensor device within the patient based on locations of the plurality of pressure sensors determined based on the received positioning signals.

At action 1104, the method 1100 may include obtaining static pressure measurements using the multiple pressure sensors of the device. The obtained static pressure measurements may include pressure measurements from at least one pressure sensor on each side of the valve. In this regard, action 1104 may include receiving, by the processing system from the plurality of pressure sensors, signals indicative of static pressure measurements obtained by the plurality of pressure sensors. The received static pressure measurements may include a first static pressure measurement obtained by a first pressure sensor positioned on a first side of a valve and a second static pressure measurement obtained by a second pressure sensor positioned on a second side of the valve opposite the first side of the valve. In some instances, the processing system may identify the pressure sensor(s) on each side of a valve based on the static pressure measurements received from the plurality of pressure sensors.

At action 1106, the method 1100 may include outputting to a display at least one of static pressure measurements associated with pressure sensors proximate the valve and/or a pressure gradient across the valve based on the static pressure measurements. In this regard, the action 1106 may include outputting, to a display in communication with the processing system, an indication of a pressure differential between the first static pressure measurement and the second static pressure measurement. The indication of the pressure differential may include a numerical value representative of a difference between the first static pressure measurement and the second static pressure measurement, which may be the determined pressure differential in some instances, and/or the first static pressure measurement and the second static pressure measurement (e.g., the associated numerical values). In some instances, the indication of the pressure differential may be color coded (e.g., green for an acceptable value, yellow for questionable value, and red for an unacceptable value).

At action 1108, the method 1100 may include diagnosing and/or treating the patient based on the static pressure measurements, pressure gradient, imaging data, and/or other data or information obtained during the procedure. The treatment may include a valve replacement (e.g., SAVR, TAVR, etc.), a transcatheter edge to edge repair (TEER), and/or other procedure in accordance with the present disclosure.

The logical operations making up the aspects of the technology described herein are referred to variously as operations, steps, objects, elements, components, or modules. Furthermore, it should be understood that these may be arranged or performed in any order, unless explicitly claimed otherwise or a specific order is inherently necessitated by the claim language. It should further be understood that the described technology may be employed in single-use and multi-use electrical and electronic devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of aspects of the present disclosure. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the present disclosure, e.g., as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. A system, comprising:
a multiple sensor device having a flexible elongate member with a distal portion sized and shaped for advancement through a vessel of a patient and at least partially through a valve of the patient, the distal portion including a plurality of pressure sensors and at least one electrode, wherein the plurality of pressure sensors are spaced apart along a length of the distal portion and wherein the at least one electrode is configured to pace a heartbeat of the patient; and
a processing system in communication with the multiple sensor device, the processing system configured to:
receive, from the plurality of pressure sensors, signals indicative of static pressure measurements obtained by the plurality of pressure sensors, wherein the static pressure measurements include a first static pressure measurement obtained by a first pressure sensor positioned on a first side of the valve and a second static pressure measurement obtained by a second pressure sensor positioned on a second side of the valve opposite the first side of the valve; and
output, to a display in communication with the processing system, an indication of a pressure differential between the first static pressure measurement and the second static pressure measurement.

2. The system of claim 1, wherein the indication of the pressure differential includes a numerical value representative of a difference between the first static pressure measurement and the second static pressure measurement.

3. The system of claim 1, wherein the indication of the pressure differential includes the first static pressure measurement and the second static pressure measurement.

4. The system of claim 1, wherein the indication of the pressure differential is color coded.

5. The system of claim 1, wherein the processing system is further configured to determine which of the plurality of pressure sensors are the first pressure sensor and the second pressure sensor based on the signals received from the plurality of pressure sensors indicative of the static pressure measurements obtained by the plurality of pressure sensors.

6. The system of claim 1, wherein the processing system is further configured to control the at least one electrode of the multiple sensor device to pace the heartbeat of the patient; and
wherein the first static pressure measurement and the second static pressure measurement are obtained during pacing of the heartbeat of the patient.

7. The system of claim 1, wherein the plurality of pressure sensors comprise optical pressure sensors.

8. The system of claim 1, wherein the plurality of pressure sensors comprise electrical pressure sensors.

9. The system of claim 1, wherein:
the plurality of pressure sensors comprise piezoelectric pressure sensors; and
the processing system is further configured to:
receive positioning signals from the plurality of pressure sensors, wherein the positioning signals are based on ultrasound positioning signals transmitted by an ultrasound imaging device;
determine, based on the received positioning signals, locations of the plurality of pressure sensors within the patient; and
output, to an imaging display in communication with the processing system, an indication of a position of the distal portion of the multiple sensor device within the patient based on the determined locations of the plurality of pressure sensors.

10. The system of claim 9, wherein the ultrasound imaging device comprises at least one of a transesophageal echocardiography (TEE) device, a transthoracic echocardiography (TTE) device, or a intracardiac echocardiography (ICE) device.

11. The system of claim 10, wherein the indication of the position of the distal portion of the multiple sensor device within the patient includes an overlay on an ultrasound image of anatomy of the patient based on ultrasound data obtained from the TEE device, the TTE device, or the ICE device.

12. The system of claim 10, wherein the processing system is further configured to output, to the imaging display, an indication of an orientation of the distal portion of the multiple sensor device within the patient based on the determined locations of the plurality of pressure sensors.

13. The system of claim 9, wherein the processing system is further configured to:
filter the signals received from the plurality of pressure sensors indicative of the static pressure measurements obtained by the plurality of pressure sensors from the positioning signals received from the plurality of pressure sensors.

14. The system of claim 1, wherein the flexible elongate member of the multiple sensor device has an outer diameter between 0.014″ and 0.47″.

15. A method, comprising:
receiving, by a processing system from a multiple sensor device positioned within a patient, positioning signals from a plurality of pressure sensors spaced apart along a length of a distal portion of the multiple sensor device, wherein the positioning signals are based on ultrasound positioning signals transmitted by an ultrasound imaging device;
outputting, to an imaging display in communication with the processing system, an indication of a position of the distal portion of the multiple sensor device within the patient based on locations of the plurality of pressure sensors determined based on the received positioning signals;
receiving, by the processing system from the plurality of pressure sensors, signals indicative of static pressure measurements obtained by the plurality of pressure sensors, the static pressure measurements including a first static pressure measurement obtained by a first pressure sensor positioned on a first side of a valve and a second static pressure measurement obtained by a second pressure sensor positioned on a second side of the valve opposite the first side of the valve; and
outputting, to a display in communication with the processing system, an indication of a pressure differential between the first static pressure measurement and the second static pressure measurement.
